(19) United States
(12) Patent Application Publication
(10) Pub. No.: US 2026/0021060 A1
(43) Pub. Date: Jan. 22, 2026

(54) READY-TO-ADMINISTER PRODUCT COMPRISING A NOREPINEPHRINE FORMULATION

(71) Applicant: Hikma Pharmaceuticals USA Inc., Berkeley Heights, NJ (US)

(72) Inventors: Martina Manenica, Zagreb (HR); Ines Berisic, Zagreb (HR); Anita Bevetek Mocnik, Zagreb (HR); Sasa Grubesic, Zagreb (HR)

(21) Appl. No.: 18/997,932

(22) PCT Filed: Jul. 21, 2023
(86) PCT No.: PCT/EP2023/070263

§ 371 Date: Jan. 23, 2025

Related U.S. Application Data
(60) Provisional application No. 63/391,911, filed on Jul. 25, 2022.

(51) Int. Cl. A61K 31/137; A61J 1/10; A61K 9/08; A61K 47/02; A61K 47/12

(52) U.S. Cl. CPC A61K 31/137; A61J 1/10; A61K 9/08; A61K 47/02; A61K 47/12

(57) ABSTRACT

Described herein are ready-to-administer products comprising liquid norepinephrine formulations contained in a flexible plastic container

### FIELD OF THE INVENTION

The present disclosure relates to a ready-to-administer product comprising a flexible plastic container and a norepinephrine formulation stored in the plastic container. Such products provide good stability over time.

### BACKGROUND

Norepinephrine is a catecholamine indicated for blood pressure control in certain acute hypotensive states (e.g., pheochromocytomectomy; sympathectomy; poliomyelitis; spinal anesthesia; myocardial infarction; septicemia; blood transfusion and drug reactions), and as an adjunct in the treatment of cardiac arrest and profound hypotension.

The structure of norepinephrine is given below.

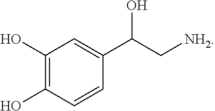 [SMILES: NCC(O)c1ccc(O)c(O)c1]

Norepinephrine is sensitive to oxidation and will normally degrade into several oxidation-related impurities. Norepinephrine is therefore often protected from oxygen by degassing the solutions used for producing the norepinephrine formulation and/or including an antioxidant in the final formulation.

Norepinephrine is also more prone to degradation when it is present in low concentrations.

One degradation impurity that can be seen in norepinephrine formulations is 1,2,3,4-tetrahydroisoquinoline-4,6,7-triol.

The structure of 1,2,3,4-tetrahydroisoquinoline-4,6,7-triol is shown below.

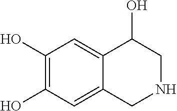 [SMILES: Oc1cc2c(cc1O)C(O)CNC2]

### SUMMARY

Described herein are ready-to-administer products comprising liquid norepinephrine formulations contained in a plastic container, wherein the ratio of the surface area of the plastic container to the volume of the formulation in the plastic container is less than 3.0 cm⁻¹.

It has been found that in a ready-to-administer product comprising a plastic container and a liquid norepinephrine formulation, wherein the ratio of the surface area of the plastic container to the volume of the formulation in the plastic container is less than 3.0 cm⁻¹, the level of 1,2,3,4-tetrahydroisoquinoline-4,6,7-triol in the formulation is minimized.

The inventors have found that the formation of 1,2,3,4-tetrahydroisoquinoline-4,6,7-triol in the product during storage can be improved by controlling the plastic container surface area in relation to volume of formulation filled into the plastic container.

Because of their enhanced storage stability, the products disclosed herein can be advantageously stored for relatively long periods of time.

It is to be understood that both the foregoing description and the following further description are exemplary and explanatory only and are not restrictive of the claims.

### DETAILED DISCLOSURE (INCLUDING DEFINITIONS)

The present disclosure relates to a ready-to-administer product comprising a plastic container and a liquid norepinephrine formulation stored in the plastic container.

Ready-to-administer means a formulation that does not need any dilution prior to administration to the patient. A ready-to-administer composition is synonymous with ready-to-infuse, ready-to-inject or ready-to-use and it is suitable to be administered directly to the patient.

As used herein, the terms “pharmaceutical composition”, “pharmaceutical formulation”, “composition” and “formulation” are used interchangeably.

By the term “aqueous solution” is understood any solution in which water is present in an amount at or above 50% v/v, such as, e.g., a solution comprising from about 50% v/v to about 100% v/v water. Accordingly, aqueous solutions include solutions comprising about 50% v/v or more, about 60% v/v or more, about 70% v/v or more, about 75% v/v or more, about 80% v/v or more, about 85% v/v or more, about 90% v/v or more, about 95% v/v or more or about 100% v/v water.

All of the numbers used herein, except for pH, are modified by the term “about.” This means that each number includes minor variations as defined +10% of the numerical value or range in question.

The term “norepinephrine” as used herein means norepinephrine or a pharmaceutically acceptable salt of norepinephrine.

In one aspect, the norepinephrine salt is norepinephrine bitartrate.

When specific amounts or ranges of amounts of norepinephrine are given in this application, all values are calculated based on norepinephrine base.

In an aspect, the concentration of norepinephrine in the liquid norepinephrine formulation is from 0.01 to 0.1 mg/ml.

In another aspect, the concentration of norepinephrine in the liquid norepinephrine formulation is 0.01 mg/ml; 0.02 mg/ml; 0.03 mg/ml; 0.04 mg/ml; 0.05mg/ml; 0.06 mg/ml; 0.07 mg/ml; 0.08 mg/ml; 0.09 mg/ml or 0.1 mg/ml.

In yet another aspect, the concentration of norepinephrine in the liquid norepinephrine formulation is 0.016 mg/ml, 0.032 mg/ml or 0.064 mg/ml.

For intravenous products to be accepted for use in humans, such products must meet the requirements of isotonicity. By the term of “isotonic” or “isotonicity” as used herein, is meant as defined in USP <785>.

The liquid norepinephrine formulation included in the ready-to-administer product optionally comprises a tonicity agent. The tonicity agent may be selected from sodium chloride, potassium chloride, mannitol, glycerin, dextrose, and mixtures thereof.

In one aspect, the tonicity agent is sodium chloride.

In an aspect, the concentration of a tonicity agent in the product is in the amount to provide an isotonic ready-to-administer product, wherein isotonicity in accordance with USP <785>.

In an aspect, norepinephrine formulation included in the ready-to-administer product has an osmolality within the physiological osmolality of blood. According to the literature, and as used herein, the physiological osmolality of blood is in the range of 270 to 340 mOsmol/kg.

In an aspect, the concentration of a tonicity agent in the norepinephrine formulation included in the ready-to-administer product should be in the amount to achieve an osmolality of the product within the targeted range of 270 to 340 mOsmol/kg.

In an aspect, the liquid norepinephrine formulation included in the ready-to-administer product is both isotonic and has an osmolality within the physiological osmolality of blood as described above.

Additionally, the liquid norepinephrine formulation included in the ready-to-administer product described herein may further comprise one or more pharmaceutical acceptable excipients such as pH adjusting agents, antioxidants, chelating agent, surfactants, complexing agents, preservatives, bulking agents, vehicles, solubilizers, thickening agents, and combinations thereof.

The pH of the liquid norepinephrine formulations is in the range from 3.0-5.0. In an aspect, the pH is in the range of 3.0-4.5. In another aspect the pH is in the range of 3.5 to 4.5 or 3.6 to 4.2. In another aspect the pH is 3.0, 3.1, 3.2, 3.3, 3.4, 3.5, 3.6, 3.7, 3.8, 3.9, 4.0, 4.1, 4.2, 4.3, 4.4 or 4.5. In yet another aspect the pH is 3.8.

“pH” is the conventional measurement unit of hydrogen ion activity in a solution at room temperature, unless another temperature is specified.

As used herein, the term “pH” in the compositions is defined as ±0.1 of the numerical value or range in question.

In an aspect, pH values are given for the formulations just after preparation, which means at the start of the shelf life.

The pH of the formulation may be adjusted in any suitable manner. The pH may be adjusted with one or more pH adjusting agents, which may be selected from mineral acids, organic acids, weak and strong bases, and salts and derivatives thereof. Examples of pH-adjusting agents include hydrochloric acid, phosphoric acid, sulfuric acid, acetic acid, succinic acid, lactic acid, citric acid, phenolic acid, sodium hydroxide, ammonium hydroxide, sodium bicarbonate, and the like, and combinations thereof.

As described herein, the ready-to-administer product comprises a plastic container that houses the liquid norepinephrine formulation. In one aspect, the plastic container can be a flexible plastic container. As used herein, the term “flexible plastic container” means flexible polymeric infusion bags or other polymeric containers produced by for example blow-fill-seal technology. Exemplary flexible plastic containers are made of polyolefins, such as polyethylene, polypropylene, copolymers and derivatives thereof, with or without other additives. An infusion bag is an example of a flexible plastic container. The available sizes of flexible plastic containers range from 50 ml to 2000 ml, such as for example 50 ml, 100 ml, 200 ml, 250 ml, 500 ml, 750 ml, 1000 ml, 1500 ml and 2000 ml.

In an aspect, a ready-to-administer product according to the present disclosure is stable at a temperature of from 2° C. to 8° C. for a certain period of time. In an aspect, a ready-to-administer product according to the present disclosure is stable under room temperature conditions for a certain period of time. By the term “room temperature” used herein, is from 20° C. to 26° C. In an aspect, a ready-to-administer product according to the present disclosure is stable at 40° C. for a certain period of time. In an aspect, the ready-to-administer product described herein is stable over time periods of 7 days (1 week), 14 days (2 weeks), 30 days (1 month), 60 days (2 months), 3 months, 4 months, 180 days (6 months), 9 months, 12 months (1 year), 14 months, 16 months, 18 months, 20 months, 24 months or more at certain specified temperature conditions.

The term “stability”, “chemical stability” or “stable” intends to mean that the product, composition or formulation exhibits an acceptable amount of norepinephrine being present, or that not more than a certain amount of norepinephrine has degraded after a certain period of time. Accordingly, in a stable product, solution or formulation, the unacceptable degradation of the norepinephrine API is avoided.

Stability as used herein includes the purity or assay of norepinephrine in a product according to the disclosure. If the product, formulation or composition initially contains norepinephrine in a certain purity or assay, the stability of the product, formulation or composition will be reflected by a decrease in the chromatographic purity or assay of norepinephrine in the product, formulation or composition over time, where a stable product, solution or composition would contain the norepinephrine in a specified chromatographic purity or assay after a predetermined time period.

For example, a stable composition can be one which has not more than 1%, 2%, 3%, 4%, 5%, 6%, 7%, 8%, 9%, 10%, of purity or assay decrease/drop of norepinephrine after a predetermined time period, determined for example by high pressure liquid chromatography using the methods described herein. This stability with regard to the overall purity or assay decrease/drop of norepinephrine after a predetermined time period may be referred to as the overall stability of the norepinephrine in the formulation.

When describing the stability of a pharmaceutical product, formulation or composition, as little degradation of the active ingredient as possible is of course an important factor. Also important are the impurities which may be formed by degradation of the active ingredient. Components of the formulation and even the container can contribute to the formation of impurities.

In the Examples, one impurity of norepinephrine identified based on its relative retention time (RRT) in the HPLC chromatogram (1,2,3,4-tetrahydroisoquinoline-4,6,7-triol) is believed to be a degradation product related to norepinephrine being in contact with the plastic material of the container housing the liquid norepinephrine formulation.

Accordingly, as an alternative to the overall stability described above, “stability” may also be defined by the amount of one or more specific impurities generated after a certain period of time. The amount of a specific impurity present may be expressed as a percentage, for example as a peak-area percentage of an HPLC chromatogram, or calculated according to a standard solution.

As used herein, when we are looking at impurity 1,2,3,4-tetrahydroisoquinoline-4,6,7-triol specifically, “stable” with regard to formation of 1,2,3,4-tetrahydroisoquinoline-4,6,7-triol is defined as no more than a 1% increase of formation of 1,2,3,4-tetrahydroisoquinoline-4,6,7-triol after a predetermined time period.

In an aspect, a stable composition can be one that has not more than 0.3%, 0.4%, 0.5%, 0.6%,.7%, 0.8%, 0.9%, 1.0%, increase in formation of 1,2,3,4-tetrahydroisoquinoline-4,6,7-triol after a predetermined time period.

In an aspect, the ready to use product is an intravenous product, that is, a product for intravenous administration of the formulation contained therein. Intravenous products generally must be sterile since the products are injected into patients. There are several ways such products can be rendered sterile; the most common ways are either by terminal sterilization of the ready-to-use product, or by sterilizing filtration of the liquid formulation through 0.2 μm filters followed by aseptic filling of the liquid formulation into pre-sterilized plastic containers.

In terminal sterilization autoclaving is normally used. In an aspect, autoclaving is a technique where the product is placed under pressurized saturated steam at up to 121 degrees C. for a period of time that can vary but would normally be up to 15 minutes, or more.

Aseptic manufacture of the sterile product means that the packaging material is sterilized and then a sterile product is filled into the packaging material under aseptic conditions. Sterilization of plastic containers can be done by different irradiation techniques for example electron beam (beta irradiation), gamma-and X-ray irradiation.

Not to be bound by any theory but the inventors believe that the formation of 1,2,3,4-tetrahydroisoquinoline-4,6,7-triol is triggered by contact of the norepinephrine in solution with the plastic material in the container. Without being held to theory, it is believed that the surface area of the plastic container is the parameter that defines the level of 1,2,3,4-tetrahydroisoquinoline-4,6,7-triol formed. It is thus believed that the surface area of the plastic will be the limiting factor for formation of 1,2,3,4-tetrahydroisoquinoline-4,6,7-triol.

Unless otherwise stated “the volume of the formulation in the plastic container” is meant to be the volume of the formulation that has been filled into the plastic container during manufacturing.

In an aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹. In an aspect the ratio of the surface area of the plastic container to the volume of the formulation in the plastic container is less than 2.0 cm⁻¹. In another aspect the ratio of the surface area of the plastic container to the volume of the formulation in the plastic container is less than 1.8 cm⁻¹. In yet another aspect the ratio of the surface area of the plastic container to the volume of the formulation in the plastic container is less than 1.6 cm⁻¹ or less than 1.5 cm⁻¹.

Unless otherwise stated the ratio of the surface area of the plastic container to the volume of the formulation filled into the plastic container is calculated as described below.

In an aspect, the ready-to-administer product comprising a plastic container and a liquid norepinephrine formulation stored in the plastic container has a ratio of the surface area of the plastic container to the volume of the formulation in the plastic container that is within the range of 0.3 cm⁻¹ to 3.0 cm⁻¹. In another aspect, the ready-to-administer product comprising a plastic container and a liquid norepinephrine formulation stored in the plastic container has a ratio of the surface area of the plastic container to the volume of the formulation in the plastic container that is within the range of 0.5 cm⁻¹ to 2.5 cm⁻¹. In yet another aspect, the ready-to-administer product comprising a plastic container and a liquid norepinephrine formulation stored in the plastic container has a ratio of the surface area of the plastic container to the volume of the formulation in the plastic container that is within the range of 1.0 cm⁻¹ to 2.0 cm⁻¹ or in the range of 1.2 cm⁻¹ to 1.8 cm⁻¹.

In an aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹, and wherein the level of 1,2,3,4-tetrahydroisoquinoline-4,6,7-triol in the formulation is less than 1.0%. In an aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹, and wherein the level of 1,2,3,4-tetrahydroisoquinoline-4,6,7-triol in the formulation is less than 0.8%. In another aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹ and wherein the level of 1,2,3,4-tetrahydroisoquinoline-4,6,7-triol in the formulation is less than 0.5%.

In another aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹, and wherein the level of 1,2,3,4-tetrahydroisoquinoline-4,6,7-triol in the formulation is less than 0.7% after storage for 3 days at 60 degrees C. In an aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹, and wherein the level of 1,2,3,4-tetrahydroisoquinoline-4,6,7-triol in the formulation is less than 0.5% after storage for 3 days at 60 degrees C. In another aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹, and wherein the level of 1,2,3,4-tetrahydroisoquinoline-4,6,7-triol in the formulation is less than 0.4% after storage for 3 days at 60 degrees C.

In an aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹, and wherein the level of 1,2,3,4-tetrahydroisoquinoline-4,6,7-triol in the formulation is less than 0.7% after storage for 1 month at 40 degrees C. In an aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹, and wherein the level of 1,2,3,4-tetrahydroisoquinoline-4,6,7-triol in the formulation is less than 0.5% after storage for 1 month at 40 degrees C. In another aspect, the ready-to-administer product comprising a plastic container and a liquid norepinephrine formulation stored in the plastic container has a ratio of the surface area of the plastic container to the volume of the formulation in the plastic container that is less than 3.0 cm⁻¹, and wherein the level of 1,2,3,4-tetrahydroisoquinoline-4,6,7-triol in the formulation is less than 0.4% after storage for 1 month at 40 degrees C.

In another aspect, the ready-to-administer product comprising a plastic container and a liquid norepinephrine formulation stored in the plastic container has a ratio of the surface area of the plastic container to the volume of the formulation in the plastic container that is less than 2.0 cm⁻¹, and wherein the level of 1,2,3,4-tetrahydroisoquinoline-4,6,7-triol in the formulation is less than 1.0%. In an aspect, the ready-to-administer product comprising a plastic container and a liquid norepinephrine formulation stored in the plastic container has a ratio of the surface area of the bag to the volume of the formulation in the plastic container that is less than 2.0 cm⁻¹, and wherein the level of 1,2,3,4-tetrahydroisoquinoline-4,6,7-triol in the formulation is less than 0.8%. In another aspect, the ready-to-administer product comprising a plastic container and a liquid norepinephrine formulation stored in the plastic container has a ratio of the surface area of the plastic container to the volume of the formulation in the plastic container that is less than 2.0 cm⁻¹, and wherein the level of 1,2,3,4-tetrahydroisoquinoline-4,6,7-triol in the formulation is less than 0.5%.

In an aspect, the ready-to-administer product comprising a plastic container and a liquid norepinephrine formulation stored in the plastic container has a ratio of the surface area of the plastic container to the volume of the formulation in the plastic container that is less than 2.0 cm⁻¹, and wherein the level of 1,2,3,4-tetrahydroisoquinoline-4,6,7-triol in the formulation is less than 0.7% after storage for 3 days at 60 degrees C. In an aspect, the ready-to-administer product comprising a plastic container and a liquid norepinephrine formulation stored in the plastic container has a ratio of the surface area of the plastic container to the volume of the formulation in the plastic container that is less than 2.0 cm⁻¹, and wherein the level of 1,2,3,4-tetrahydroisoquinoline-4,6,7-triol in the formulation is less than 0.5% after storage for 3 days at 60 degrees C. In another aspect, the ready-to-administer product comprising a plastic container and a liquid norepinephrine formulation stored in the plastic container has a ratio of the surface area of the plastic container to the volume of the formulation in the plastic container that is less than 2.0 cm⁻¹, and wherein the level of 1,2,3,4-tetrahydroisoquinoline-4,6,7-triol in the formulation is less than 0.4% after storage for 3 days at 60 degrees C.

In an aspect, the ready-to-administer product comprising a plastic container and a liquid norepinephrine formulation stored in the plastic container has a ratio of the surface area of the plastic container to the volume of the formulation in the plastic container that is less than 2.0 cm⁻¹, and wherein the level of 1,2,3,4-tetrahydroisoquinoline-4,6,7-triol in the formulation is less than 0.7% after storage for 1 month at 40 degrees C. In an aspect, the ready-to-administer product comprising a plastic container and a liquid norepinephrine formulation stored in the plastic container has a ratio of the surface area of the plastic container to the volume of the formulation in the plastic container that is less than 2.0 cm⁻¹, and wherein the level of 1,2,3,4-tetrahydroisoquinoline-4,6,7-triol in the formulation is less than 0.5% after storage for 1 month at 40 degrees C. In another aspect, the ready-to-administer product comprising a plastic container and a liquid norepinephrine formulation stored in the plastic container has a ratio of the surface area of the plastic container to the volume of the formulation in the plastic container that is less than 2.0 cm⁻¹, and wherein the level of 1,2,3,4-tetrahydroisoquinoline-4,6,7-triol in the formulation is less than 0.3% after storage for 1 month at 40 degrees C.

In another aspect, the ready-to-administer product comprising a plastic container and a liquid norepinephrine formulation stored in the plastic container has a ratio of the surface area of the plastic container to the volume of the formulation in the plastic container that is less than 1.8 cm⁻¹, and wherein the level of 1,2,3,4-tetrahydroisoquinoline-4,6,7-triol in the formulation is less than 1.0%. In an aspect, the ready-to-administer product comprising a plastic container and a liquid norepinephrine formulation stored in the plastic container has a ratio of the surface area of the plastic container to the volume of the formulation in the plastic container that is less than 1.8 cm⁻¹, and wherein the level of 1,2,3,4-tetrahydroisoquinoline-4,6,7-triol in the formulation is less than 0.8%. In another aspect, the ready-to-administer product comprising a plastic container and a liquid norepinephrine formulation stored in the plastic container has a ratio of the surface area of the plastic container to the volume of the formulation in the plastic container that is less than 1.8 cm⁻¹ and wherein the level of 1,2,3,4-tetrahydroisoquinoline-4,6,7-triol in the formulation is less than 0.5%.

In an aspect, the ready-to-administer product comprising a plastic container and a liquid norepinephrine formulation stored in the plastic container has a ratio of the surface area of the bag to the volume of the formulation in the plastic container that is less than 1.8 cm⁻¹, and wherein the level of 1,2,3,4-tetrahydroisoquinoline-4,6,7-triol in the formulation is less than 0.7% after storage for 3 days at 60 degrees C. In an aspect, the ready-to-administer product comprising a plastic container and a liquid norepinephrine formulation stored in the plastic container has a ratio of the surface area of the plastic container to the volume of the formulation in the plastic container that is less than 1.8 cm⁻¹, and wherein the level of 1,2,3,4-tetrahydroisoquinoline-4,6,7-triol in the formulation is less than 0.5% after storage for 3 days at 60 degrees C. In another aspect, the ready-to-administer product comprising a plastic container and a liquid norepinephrine formulation stored in the plastic container has a ratio of the surface area of the plastic container to the volume of the formulation in the plastic container that is less than 1.8 cm⁻¹, and wherein the level of 1,2,3,4-tetrahydroisoquinoline-4,6,7-triol in the formulation is less than 0.4% after storage for 3 days at 60 degrees C.

In an aspect, the ready-to-administer product comprising a plastic container and a liquid norepinephrine formulation stored in the plastic container has a ratio of the surface area of the plastic container to the volume of the formulation in the plastic container that is less than 1.8 cm⁻¹, and wherein the level of 1,2,3,4-tetrahydroisoquinoline-4,6,7-triol in the formulation is less than 0.7% after storage for 1 month at 40 degrees C. In an aspect, the ready-to-administer product comprising a plastic container and a liquid norepinephrine formulation stored in the plastic container has a ratio of the surface area of the plastic container to the volume of the formulation in the plastic container that is less than 1.8 cm⁻¹, and wherein the level of 1,2,3,4-tetrahydroisoquinoline-4,6,7-triol in the formulation is less than 0.5% after storage for 1 month at 40 degrees C. In another, aspect the ready-to-administer product comprising a plastic container and a liquid norepinephrine formulation stored in the plastic container has a ratio of the surface area of the plastic container to the volume of the formulation in the plastic container that is less than 1.8 cm⁻¹, and wherein the level of 1,2,3,4-tetrahydroisoquinoline-4,6,7-triol in the formulation is less than 0.3% after storage for 1 month at 40 degrees C.

In another aspect, the ready-to-administer product comprising a plastic container and a liquid norepinephrine formulation stored in the plastic container has a ratio of the surface area of the plastic container to the volume of the formulation in the plastic container that is less than 1.6 cm⁻¹, and wherein the level of 1,2,3,4-tetrahydroisoquinoline-4,6,7-triol in the formulation is less than 1.0%. In an aspect, the ready-to-administer product comprising a plastic container and a liquid norepinephrine formulation stored in the plastic container has a ratio of the surface area of the plastic container to the volume of the formulation in the plastic container that is less than 1.6 cm⁻¹, and wherein the level of 1,2,3,4-tetrahydroisoquinoline-4,6,7-triol in the formulation is less than 0.8%. In another aspect, the ready-to-administer product comprising a plastic container and a liquid norepinephrine formulation stored in the plastic container has a ratio of the surface area of the plastic container to the volume of the formulation in the plastic container that is less than 1.6 cm⁻¹, and wherein the level of 1,2,3,4-tetrahydroisoquinoline-4,6,7-triol in the formulation is less than 0.5%.

In an aspect, the ready-to-administer product comprising a plastic container and a liquid norepinephrine formulation stored in the plastic container has a ratio of the surface area of the plastic container to the volume of the formulation in the flexible plastic container that is less than 1.6 cm⁻¹, and wherein the level of 1,2,3,4-tetrahydroisoquinoline-4,6,7-triol in the formulation is less than 0.7% after storage for 3 days at 60 degrees C. In an aspect, the ready-to-administer product comprising a plastic container and a liquid norepinephrine formulation stored in the plastic container has a ratio of the surface area of the plastic container to the volume of the formulation in the plastic container that is less than 1.6 cm⁻¹, and wherein the level of 1,2,3,4-tetrahydroisoquinoline-4,6,7-triol in the formulation is less than 0.5% after storage for 3 days at 60 degrees C. In another aspect, the ready-to-administer product comprising a plastic container and a liquid norepinephrine formulation stored in the plastic container has a ratio of the surface area of the plastic container to the volume of the formulation in the plastic container that is less than 1.6 cm⁻¹, and wherein the level of 1,2,3,4-tetrahydroisoquinoline-4,6,7-triol in the formulation is less than 0.4% after storage for 3 days at 60 degrees C.

In another aspect, the ready-to-administer product comprising a plastic container and a liquid norepinephrine formulation stored in the plastic container has a ratio of the surface area of the plastic container to the volume of the formulation in the plastic container that is less than 1.6 cm⁻¹, and wherein the level of 1,2,3,4-tetrahydroisoquinoline-4,6,7-triol in the formulation is less than 0.7% after storage for 1 month at 40 degrees C. In an aspect, the ready-to-administer product comprising a plastic container and a liquid norepinephrine formulation stored in the plastic container has a ratio of the surface area of the plastic container to the volume of the formulation in the plastic container that is less than 1.6 cm⁻¹, and wherein the level of 1,2,3,4-tetrahydroisoquinoline-4,6,7-triol in the formulation is less than 0.5% after storage for 1 month at 40 degrees C. In another aspect, the ready-to-administer product comprising a plastic container and a liquid norepinephrine formulation stored in the plastic container has a ratio of the surface area of the plastic container to the volume of the formulation in the plastic container that is less than 1.6 cm⁻¹, and wherein the level of 1,2,3,4-tetrahydroisoquinoline-4,6,7-triol in the formulation is less than 0.3% after storage for 1 month at 40 degrees C.

In an aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 1.0%. In an aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.8%. In another aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.5%.

In an aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.7% after storage for 3 days at 60 degrees C. In an aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.5% after storage for 3 days at 60 degrees C. In another aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.4% after storage for 3 days at 60 degrees C.

In another aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.7% after storage for 1 month at 40 degrees C. In an aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.5% after storage for 1 month at 40 degrees C. In another aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.4% after storage for 1 month at 40 degrees C.

In an aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.6% after storage for 2 months at 25 degrees C. In an aspect, the ready-to-administer product comprising a plastic container and a liquid norepinephrine formulation stored in the plastic container has a ratio of the surface area of the plastic container to the volume of the formulation in the plastic container that is less than 1.5 cm⁻¹, and wherein the level of 1,2,3,4-tetrahydroisoquinoline-4,6,7-triol in the formulation is less than 0.5% after storage for 2 months at 25 degrees C. In another aspect, the ready-to-administer product comprising a plastic container and a liquid norepinephrine formulation stored in the plastic container has a ratio of the surface area of the plastic container to the volume of the formulation in the flexible plastic container that is less than 1.5 cm⁻¹, and wherein the level of 1,2,3,4-tetrahydroisoquinoline-4,6,7-triol in the formulation is less than 0.4% after storage for 2 months at 25 degrees C.

In an aspect, the ready-to-administer product comprising a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.6% after storage for 2 months at 25 degrees C. In an aspect, the ready-to-administer product comprising a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.5% after storage for 2 months at 25 degrees C. In another aspect, the ready-to-administer product comprising a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.4% after storage for 2 months at 25 degrees C.

In an aspect, the ready-to-administer product comprising a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.6% after storage for 2 months at 25 degrees C. In an aspect, the ready-to-administer product comprising a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.5% after storage for 2 months at 25 degrees C. In another aspect, the ready-to-administer product comprising a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.4% after storage for 2 months at 25 degrees C.

In an aspect, the ready-to-administer product consists essentially of a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.6% after storage for 2 months at 25 degrees C. In an aspect, the ready-to-administer product consists essentially of a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.5% after storage for 2 months at 25 degrees C. In another aspect, the ready-to-administer product consists essentially of a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.4% after storage for 2 months at 25 degrees C.

In an aspect, the ready-to-administer product consists essentially of a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.6% after storage for 2 months at 25 degrees C. In an aspect, the ready-to-administer product consists essentially of a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.5% after storage for 2 months at 25 degrees C. In another aspect, the ready-to-administer product consists essentially of a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.4% after storage for 2 months at 25 degrees C.

In an aspect, the ready-to-administer product consists of a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.6% after storage for 2 months at 25 degrees C. In an aspect, the ready-to-administer product consists of a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.5% after storage for 2 months at 25 degrees C. In another aspect, the ready-to-administer product consists of a plastic container and a liquid norepinephrine formulation stored in the plastic container has a ratio of the surface area of the plastic container to the volume of the formulation in the plastic container that is in the range of 1.2 cm⁻¹ to 1.8 cm⁻¹, and wherein the level of 1,2,3,4-tetrahydroisoquinoline-4,6,7-triol in the formulation is less than 0.4% after storage for 2 months at 25 degrees C.

In an aspect, the ready-to-administer product consists of a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.6% after storage for 2 months at 25 degrees C. In an aspect, the ready-to-administer product consists of a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.5% after storage for 2 months at 25 degrees C. In another aspect, the ready-to-administer product consists of a plastic container and a liquid norepinephrine formulation stored in the plastic container has a ratio of the surface area of the plastic container to the volume of the formulation in the plastic container that is in the range of 1.0 cm⁻¹ to 2.0 cm⁻¹, and wherein the level of 1,2,3,4-tetrahydroisoquinoline-4,6,7-triol in the formulation is less than 0.4% after storage for 2 months at 25 degrees C.

In one aspect, the plastic container may further be overwrapped. In one aspect, the overwrap may comprise a gas-barrier and/or a light barrier material.

In one aspect, the space between the container and the overwrap is filled with an inert gas or any other suitable gas. In one aspect, the space between the container and the overwrap is filled with nitrogen, argon, or any combination thereof.

In one aspect, the space between the container and the overwrap is under vacuum.

In one aspect, the space between the container and overwrap comprises an oxygen scavenger.

In one aspect, the ready-to-administer product may be used for controlling blood pressure in certain acute hypotensive states (e.g., pheochromocytomectomy; sympathectomy; poliomyelitis; spinal anesthesia; myocardial infarction; septicaemia; blood transfusion and drug reactions).

In one aspect, the ready-to-administer product is used to increase the blood pressure in adults with clinically important hypotension resulting primarily from vasodilation in the settings of anesthesia and septic shock.

In one aspect, the ready-to-administer product may be used as an adjunct in the treatment of cardiac arrest and profound hypotension.

### FIGURES

FIG. 1 shows where to measure the x and y lengths of the rectangular flexible plastic container, e.g. bag, to be used in the calculation of the Surface Area (SA) of the bag.

FIG. 2 shows the results from the three bags with a low surface area to volume filled ratio compared to bags with a high surface area to volume filled ratio based on data shown in Table 1.

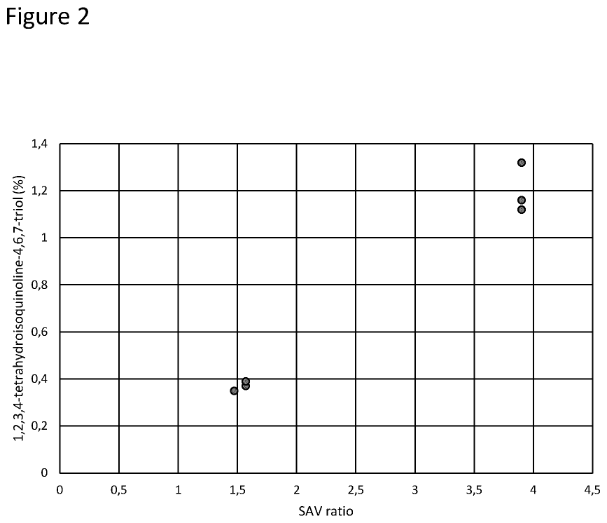

FIG. 3 shows the area % of 1,2,3,4-tetrahydroisoquinoline-4,6,7-triol relative to the concentration of norepinephrine based on data shown in Table 4.

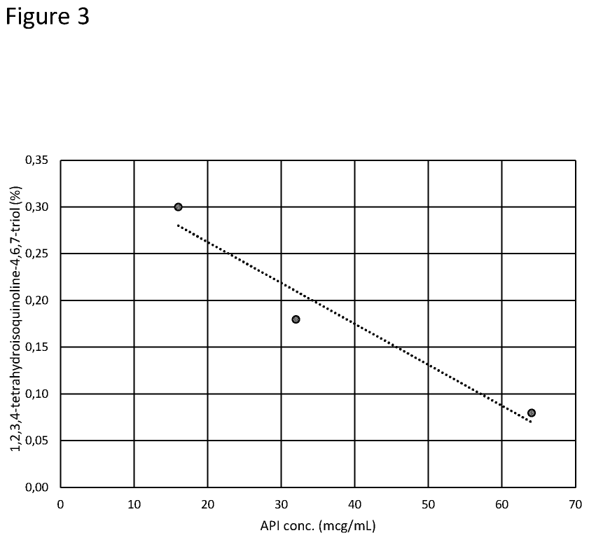

### EXAMPLES (METHODS, RESULTS, DISCUSSION)

### Hplc Assay Method

Mobile phase A: 1,13 g Sodium 1-heptanesulfonate+900 ml H₂O+100 ml MeOH, to pH 3±0.2 with H₃PO₄

Mobile phase B: Methanol

The gradient of mobile phases is shown below.

| Time | Mobile | Mobile |
|---|---|---|
| [min] | phase A % | phase B % |
| 0 | 100 | 0 |
| 15 | 100 | 0 |
| 25 | 80 | 20 |
| 40 | 80 | 20 |
| 40.5 | 100 | 0 |
| 50 | 100 | 0 |

Standard solution: 0.032 mg/ml of norepinephrine bitartrate monohydrate in water (equivalent to 0.016 mg/ml norepinephrine base).

Sample solution: Nominally 0.032 mg/ml of norepinephrine bitartrate monohydrate (equivalent to 0.016 mg/ml norepinephrine base).

### CHROMATOGRAPHIC SYSTEM

Mode: LC

Detector: UV 215 nm.

Column: 4.6×25 cm; 5 μm packing L1

Column temperature: 25° C.

Flow rate: 1.0 mL/min

### CALCULATION

Calculate the percentage of the labeled amount of norepinephrine.

$$Assay \% = ( \frac{{A}_{s}}{{A}_{std}} ) \times ( \frac{{c}_{std}}{{c}_{s}} ) \times 100$$

Aₛ-peak response of norepinephrine from the Sample solution

A_{std}-peak response of norepinephrine from the Standard solution

C_{std}-concentration of norepinephrine base in the Standard solution (mg/ml)

Cₛ-nominal concentration of norepinephrine in the sample solution (mg/ml)

### HPLC IMPURITIES METHOD

Mobile phase A, Mobile phase B and gradient of mobile phases are the same as mentioned above in the HPLC Assay method.

The Chromatographic system is the same a mentioned above in the HPLC Assay method.

### CALCULATION

The content of impurities is given as area % of the total area, calculated using the following equation:

$$Impurity \% = ( \frac{{A}_{i}}{{A}_{t o t}} ) \times 100$$

Impurity %=area % of an individual peak

Aᵢ=peak area of an individual peak

Aₜₒₜ=total sample peak area

Surface area to Volume ratio:

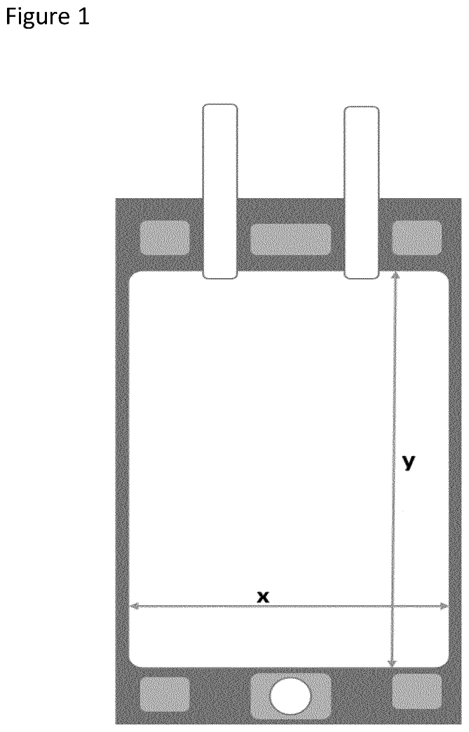

The surface area to volume (herein also referred to as SA:V or SAV) ratio is calculated by dividing the inner surface area of the container with “the volume of the formulation in the plastic container”. The inner surface area of a container equals the total usable inner surface area of the container that is available for containing the solution (i.e. the parts of the container used for solution transfer or delivery are not to be considered even if they possibly can come in direct contact with solution, such as tubes, ports, stoppers, delivery sets and similar). The inner surface area of a container can be calculated by simple mathematical calculations from known dimensions of the container, depending on the shape of the container. In an example of a rectangular flexible bag, the inner surface area of a container is made of two sides. To calculate the total inner Surface Area (SA) of a rectangular bag, x and y was measured prior to filling the bag, as shown in FIG. 1, and the Surface Area was calculated by the following formula: SA=2xy

The ratio between the Surface Area and the Volume was calculated by the following formula: Surface Area to Volume Ratio=SA/volume of the solution filled into the bag

| | V (fill)/ | | | SA = | ratio |
|---|---|---|---|---|---|
| Bag no. | cm³ | x/cm | y/cm | 2xy/cm² | (SA:V)(cm⁻¹) |
| 1 | 250 | 11.5 | 16 | 368 | 1.47 |
| 2 | 200 | 9.5 | 16.5 | 313.5 | 1.57 |
| 3 | 200 | 9.5 | 16.5 | 313.5 | 1.57 |
| 4 | 50 | 9.2 | 10.6 | 195.04 | 3.90 |
| 5 | 50 | 9.2 | 10.6 | 195.04 | 3.90 |
| 6 | 50 | 9.2 | 10.6 | 195.04 | 3.90 |

### Preparation of Exemplary Norepinephrine Formulation:

Norepinephrine was dissolved in water for injection to a concentration of 0.8 to 2 mg/ml to prepare a concentrated solution of norepinephrine. A second solution was prepared by dissolving the selected excipients; sodium chloride and citric acid, in water for injection. The solution was purged with nitrogen to remove oxygen from the solution. The pH was adjusted with sodium hydroxide to provide a target pH within the range of 3.0-5.0. Norepinephrine was added into the second solution by adding amounts of the concentrated norepinephrine solution to provide a final norepinephrine concentration of 0.01 to 0.1 mg/ml. The solutions were mixed, and the volume was made up to a final volume by adding water. The final norepinephrine solution was filtered through a 0.2 um filter and was aseptically filled directly into the flexible plastic container and the container was sealed. The filled container was placed in an aluminum overwrap and an oxygen scavenger was included between the container and the overwrap.

### RESULTS

### Example 1

All bags were sterilized using beta irradiation techniques. The bags were aseptically filled with an aqueous norepinephrine solution comprising 0.016 mg norepinephrine/ml and having a pH about 3.5. The bags were overwrapped, and an oxygen scavenger was included between the bag and the overwrap.

The amount of 1,2,3,4-tetrahydroisoquinoline-4,6,7-triol was measured after storage for 3 days at 60 degrees C. The results are shown in Table 1.

TABLE 1

| | Volume | | | | 1,2,3,4- |
|---|---|---|---|---|---|
| Bag | filled | | SA | SAV ratio | tetrahydroisoquinoline- |
| no. | (cm³) | | (cm²) | (cm⁻¹⁾ | 4,6,7-triol |
| 1 | 250 | mL | 368 | 1.47 | 0.35 |
| 2 | 200 | mL | 313.5 | 1.57 | 0.37 |
| 3 | 200 | mL | 313.5 | 1.57 | 0.39 |
| 4 | 50 | mL | 195.04 | 3.9 | 1.12 |
| 5 | 50 | mL | 195.04 | 3.9 | 1.32 |
| 6 | 50 | mL | 195.04 | 3.9 | 1.16 |

The amount of 1,2,3,4-tetrahydroisoquinoline-4,6,7-triol was measured after storage for 1 month, 2 months and 6 months at 40 degrees C. The results are shown in Table 2.

TABLE 2

| | | | | | | | 1,2,3,4- |
|---|---|---|---|---|---|---|---|
| | Volume | | | | | | tetrahydro- |
| Bag | filled | | SA | SAV ratio | Time | | isoquinoline- |
| no. | (cm³) | | (cm²) | (cm⁻¹) | point | | 4,6,7-triol |
| 1 | 250 | mL | 368 | 1.47 | 1 | month | 0.32 |
| 4 | 50 | mL | 195.04 | 3.9 | 1 | month | 1.23 |
| 3 | 200 | mL | 313.5 | 1.57 | 2 | months | 0.72 |
| 4 | 50 | mL | 195.04 | 3.9 | 2 | months | 1.54 |
| 3 | 200 | mL | 313.5 | 1.57 | 6 | months | 0.88 |
| 4 | 50 | mL | 195.04 | 3.9 | 6 | months | 1.61 |

The amount of 1,2,3,4-tetrahydroisoquinoline-4,6,7-triol was measured after storage for 1 month, 2 months, 3 months, 6 months and 12 months at 25 degrees C. The results are shown in Table 3.

| | | | | | | | 1,2,3,4- |
|---|---|---|---|---|---|---|---|
| | Volume | | | | | | tetrahydro- |
| Bag | filled | | SA | SAV ratio | | | isoquinoline- |
| no. | (cm³) | | (cm²) | (cm⁻¹) | Time point | | 4,6,7-triol |
| 3 | 200 | mL | 368 | 1.47 | 1 | month | 0.16 |
| 4 | 50 | mL | 195.04 | 3.9 | 1 | month | 0.30 |
| 3 | 200 | mL | 368 | 1.47 | 2 | months | 0.25 |
| 4 | 50 | mL | 195.04 | 3.9 | 2 | months | 0.53 |
| 3 | 200 | mL | 368 | 1.47 | 3 | months | 0.35 |
| 4 | 50 | mL | 195.04 | 3.9 | 3 | months | 0.76 |
| 3 | 200 | mL | 368 | 1.47 | 6 | months | 0.54 |
| 4 | 50 | mL | 195.04 | 3.9 | 6 | months | 1.17 |
| 3 | 200 | mL | 368 | 1.47 | 12 | months | 0.75 |
| 4 | 50 | mL | 195.04 | 3.9 | 12 | months | 1.30 |

### Example 2

All container bags were sterilized using beta irradiation techniques. The bags were aseptically filled with an aqueous norepinephrine solution comprising three different concentrations of norepinephrine: 0.016 mg/ml, 0.032 mg/ml and 0.064 mg/ml and having a pH about 3.5. The bags were overwrapped, and an oxygen scavenger was included between the bag and the overwrap.

The amount of 1,2,3,4-tetrahydroisoquinoline-4,6,7-triol was measured after storage for 3 days at 60 degrees C. The results are shown in Table 4.

TABLE 4

| | Volume | | | 1,2,3,4- |
|---|---|---|---|---|
| API conc. | filled | SA | SAV ratio | tetrahydroisoquinoline- |
| mg/mL | (cm³) | (cm²) | (cm⁻¹) | 4,6,7-triol |
| 0.016 | 250 | 368 | 1.47 | 0.30 |
| 0.032 | 250 | 368 | 1.47 | 0.18 |
| 0.064 | 250 | 368 | 1.47 | 0.08 |

The amount of 1,2,3,4-tetrahydroisoquinoline-4,6,7-triol was measured after storage for 3 months and 6 months at 40 degrees C. The results are shown in Table 5.

TABLE 5

| | Volume | | | 1,2,3,4- | |
|---|---|---|---|---|---|
| API conc. | filled | SA | SAV ratio | tetrahydroisoquinoline- | |
| mg/mL | (cm³) | (cm²) | (cm⁻¹) | 4,6,7-triol | |
| 0.016 | 250 | 368 | 1.47 | 3 months | 0.30 |
| | | | | 6 months | 0.63 |
| 0.032 | 250 | 368 | 1.47 | 3 months | 0.22 |
| | | | | 6 months | 0.28 |
| 0.064 | 250 | 368 | 1.47 | 3 months | 0.12 |
| | | | | 6 months | 0.16 |

The amount of 1,2,3,4-tetrahydroisoquinoline-4,6,7-triol was measured after storage for 3 months, 6 months and 12 months at 25 degrees C. The results are shown in Table 6.

TABLE 6

| | Volume | | | 1,2,3,4- | |
|---|---|---|---|---|---|
| API conc. | filled | SA | SAV ratio | tetrahydroisoquinoline- | |
| mg/mL | (cm³) | (cm²) | (cm⁻¹) | 4,6,7-triol | |
| 0.016 | 250 | 368 | 1.47 | 3 months | 0.19 |
| | | | | 6 months | 0.22 |
| | | | | 12 months | 0.32 |
| 0.032 | 250 | 368 | 1.47 | 3 months | 0.11 |
| | | | | 6 months | 0.14 |
| | | | | 12 months | 0.18 |
| 0.064 | 250 | 368 | 1.47 | 3 months | 0.05 |
| | | | | 6 months | 0.08 |
| | | | | 12 months | 0.08 |

1. A ready-to-administer product comprising a plastic container and a liquid formulation stored in the plastic container,
wherein the liquid formulation comprises norepinephrine, and
wherein the ratio of the inner surface area of the plastic container to the volume of the liquid formulation is less than 3.0 cm⁻¹ and wherein the amount of 1,2,3,4-tetrahydroisoquinoline-4,6,7-triol is less than 0.5% as measured by HPLC after storage for 2 months at 25 degrees C.

2. The product according to claim 1, wherein the concentration of norepinephrine is in the range of 0.01 mg/ml to 0.1 mg/ml.

3. The product according to claim 1, wherein the concentration of norepinephrine is 0.016 mg/ml, 0.032 mg/ml or 0.064 mg/ml.

4. The product according to claim 1, wherein the liquid formulation comprises a tonicity agent.

5. The product according to claim 1, wherein the liquid formulation comprises a tonicity agent selected from the group consisting of sodium chloride, potassium chloride, mannitol, glycerin, dextrose, and mixtures of any of these.

6. The product according to claim 1, the pH of the liquid formulation is in the range of 3.0 to 5.0.

7. The product according to claim 1, wherein the pH of the liquid formulation is in the range of 3.5 to 4.5.

8. The product according to claim 1, wherein the ratio of the surface area of the container to the volume of the liquid formulation is in the range of 0.3 cm⁻¹ to 3.0 cm⁻¹.

9. The product according to claim 1, wherein the ratio of the surface area of the container to the volume of the liquid formulation is in the range of 0.5 cm⁻¹ to 2.5 cm⁻¹.

10. The product according to claim 1, wherein the ratio of the surface area of the container to the volume of the liquid formulation is in the range of 1.0 cm⁻¹ to 2.0 cm⁻¹.

11. The product according to claim 1, wherein the ratio of the surface area of the container to the volume of the liquid formulation is in the range of 1.2 cm⁻¹ to 1.8 cm⁻¹.

12. The product according to claim 1, wherein the liquid formulation is an aqueous formulation.

13. The product according to claim 1, wherein the liquid formulation does not contain a sulfite antioxidant.

14. The product according to claim 1, wherein the inner surface of the plastic container comprises polyolefins.

15. The product according to claim 1, wherein the plastic container is a flexible plastic container.

16. The product according to any one of claim 1, wherein the liquid formulation is an isotonic aqueous solution with pH in the range of 3.5 to 4.5 comprising norepinephrine bitartrate and citric acid, wherein the formulation comprises 0.01 mg/ml to 0.1 mg/ml norepinephrine, wherein the container is ready-to-administer product in the form of an infusion bag comprising 50 to 500 ml of the liquid formulation, wherein the inner surface of the infusion bag comprises polyolefins, and wherein the ratio of the inner surface area of the container to the volume of the formulation is in the range of 1.0 cm⁻¹ to 2.0 cm⁻¹.

17. The product according to claim 16, wherein the amount of 1,2,3,4-tetrahydroisoquinoline-4,6,7-triol is less than 0.5% as measured by HPLC after storage for 2 months at 25 degrees C.

18. The product according to claim 1, produced by aseptic filling of the liquid formulation into plastic containers pre-sterilized by irradiation.

19. The product according to claim 18, wherein the irradiation is beta-irradiation, gamma-irradiation or X-ray irradiation.

20. The product according to claim 1, wherein the product is sterilized by autoclaving.

21. A method to increase the blood pressure in adults with clinically important hypotension resulting primarily from vasodilation in the settings of anesthesia and septic shock comprising administering the product according to claim 1 to a patient in need thereof.
